(19) United States
(12) Patent Application Publication
(10) Pub. No.: US 2024/0282455 A1
(43) Pub. Date: Aug. 22, 2024

(54) METHODS, SYSTEMS, AND DEVICES FOR ANALYZING OBJECTIVE HEALTH DATA AND SUBJECT-CONVEYED HEALTH INFORMATION FOR HEALTH INFORMATION REPORTING, TREATMENT PLANNING, DIAGNOSES, AND TREATMENT

(71) Applicant: University of Maryland, Baltimore County, Baltimore, MD (US)

(72) Inventors: Justin Brooks, Fall Church, VA (US); Jan Ver Eecke, Potomac, MD (US); Sarah Conklin, Baltimore, MD (US); Jessica Dietch, Baltimore, MD (US)

(21) Appl. No.: 18/583,009

(22) Filed: Feb. 21, 2024

Related U.S. Application Data
(60) Provisional application No. 63/447,316, filed on Feb. 21, 2023.

(51) Int. Cl. G16H 50/30; G16H 40/67

(52) U.S. Cl. CPC G16H 50/30; G16H 40/67

(57) ABSTRACT

Methods, systems, and devices for analyzing objective health data and subject-conveyed health information are disclosed for health information reporting, treatment planning, diagnoses, and treatment. According to an aspect, a method can include receiving objective health data of a subject. Further, the method can include receiving a first set of information about the subject's health. The first set of information was conveyed by the subject. The method includes presenting the objective health data to the subject. The method includes receiving, subsequent to presentation of the objective health data to the subject, a second set of information about the subject's health, wherein the second set of information was conveyed by the subject. The method can also include analyzing the objective health data, the first set of information, and the second set of information. Further, the method can include determining a treatment plan, diagnosis, and/or health information based on the analysis.

### CROSS REFERENCE TO RELATED APPLICATION

This application claims priority to U.S. Provisional Patent Application No. 63/447,316, filed Feb. 21, 2023, and titled “Method and System to Improve the Accuracy of Patient Reported Outcomes in Clinical Trials”, the content of which is incorporated herein by reference in its entirety.

### TECHNICAL FIELD

The presently disclosed subject matter relates generally to health data analyses, reporting and diagnoses. Particularly, the presently disclosed subject matter relates to methods, systems, and devices for analyzing objective health data and subject-conveyed health information for health information reporting, treatment planning, diagnoses, and treatment.

### BACKGROUND

A major problem with current methods of measuring patient reported outcomes (PROs) is that they are highly dependent on the patient's memory which confounds the treatment effect. Patients as humans, have fallible, pliable, and plastic memories that make their utility in determining a treatment effect highly questionable. These sources of variability accumulate over time resulting in significant uncertainty that arises from response shifting, multiple sources of bias, recency effect, and variable recall. Numerous randomized, controlled clinical trials rely on PRO endpoints for FDA approval that are typically captured through survey and attempt to quantify patients' symptoms. PROs have become increasingly prevalent in clinical research and can be used to measure multiple clinical entities including headache, nausea, itch, pain, quality of life (QOL) among several others. Across indications, the reliance on human memory makes interpretation of treatment effects exceptionally complex and often leads to negative results. Consequently, medications that could potentially benefits large number of patients are abandoned because they do not meet subjective primary endpoints. One of the central issues with PRO measurement is that they rely on subjective data that is generated by human memory which is inherently fallible, a phenomenon which is well understood in judicial processes.

In some cases, polysomnography (PSG) and actigraphy have been the traditional methods for objective/inferred sleep assessment; however, consumer wearable devices are becoming increasingly popular as a means of collecting out-of-lab sensor-based sleep data. Compared to PSG, sensor-based wearables are more ecologically valid, user-friendly, accessible, and affordable. Unlike actigraphy, recent-generation wearables have sleep-stage capabilities and can provide real-time feedback and personalized recommendation to users based on sleep patterns. Yet, the validity and reliability of sleep data derived from the devices which use differing sensors and algorithms that are often proprietary remain an enduring issue with wearables/sensor-based devices.

There is a continuing need to utilize available health data and information for improving treatment planning, reporting, treatment and diagnoses. For example, it is desired to better utilize objective health data, such as data acquired by physical sensors, and self-reported health information.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is a block diagram of a system for analyzing objective health data and subject-conveyed health information for health information reporting, treatment planning, and diagnoses in accordance with embodiments of the present disclosures;

FIG. 2 is a method of analyzing objective health data and subject-conveyed health information for health information reporting, treatment planning, and diagnoses in accordance with embodiments of the present disclosure;

FIG. 3 are graphs showing data distributions of self-reported data for sleep parameters;

FIG. 4 are graphs showing Bayesian probability of direction and possible parameter values;

FIG. 5 are graphs showing Bayesian probability of direction and possible parameter values for confidence and quality appraisals;

FIG. 6 are graphs showing Bland Altman agreement analysis for sleep onset latency;

FIG. 7 are graphs showing Bland Altman agreement analysis for total sleep time;

FIG. 8 are graphs showing Binomial proportions test for sleep parameters;

FIG. 9 is a block diagram of another system for analyzing objective health data and subject-conveyed health information for health information reporting, treatment planning, and diagnoses in accordance with embodiments of the present disclosures;

FIG. 10A is an image showing various soft goods for the device including breathable washable neoprene, a flexible plastic mold of the shin and a pocket for the electronics module;

FIG. 10B is an image showing contents of the electronics module including the 3-LED photoplethysmography (PPG) sensor, thermistor, battery, charge circuitry, and processor;

FIG. 11 is a schematic of an example system for assisting subjects to contextualize their responses in accordance with embodiments of the presented disclosure;

FIG. 12 is a high-level schematic of a system in accordance with embodiments of the present disclosure;

FIG. 13 is an image of an example survey for a subject in accordance with embodiments of the present disclosure; and

FIG. 14 is a flow diagram of a method of use for health information reporting, treatment planning, diagnoses, and treatment in the context of sleep in accordance with embodiments of the present disclosure.

### SUMMARY

The presently disclosed subject matter relates to methods, systems, and devices for analyzing objective health data and subject-conveyed health information for health information reporting, treatment planning, diagnoses, and treatment. According to an aspect, a method may be implemented at a computing device having a health data manager. The method can include receiving objective health data of a subject. Further, the method can include receiving a first set of information about the subject's health, wherein first set of information was conveyed by the subject. The method can also include presenting the objective health data to the subject. Further, the method can include receiving, subsequent to presentation of the objective health data to the subject, a second set of information about the subject's health, wherein the second set of information was conveyed by the subject. The method can also include analyzing the objective health data, the first set of information, and the second set of information. Further, the method can include determining a treatment plan, diagnosis, and/or health information based on the analysis. The method can also include presenting, via a user interface, the treatment plan, diagnosis, and/or health information.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as between 1%-50%, it is intended that values such as between 2%-40%, 10%-30%, or 1%-3%, etc. are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

As referred to herein, the terms “computing device” and “entities” should be broadly construed and should be understood to be interchangeable. They may include any type of computing device, for example, a server, a desktop computer, a laptop computer, a smart phone, a cell phone, a pager, a personal digital assistant (PDA, e.g., with GPRS NIC), a mobile computer with a smartphone client, or the like.

As used herein, the term “memory” is generally a storage device of a computing device. Examples include, but are not limited to, read-only memory (ROM) and random access memory (RAM).

As referred to herein, the term “user interface” is generally a system by which users interact with a computing device. A user interface can include an input for allowing users to manipulate a computing device, and can include an output for allowing the system to present information and/or data, indicate the effects of the user's manipulation, etc. An example of a user interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. A user interface can include an input for allowing users to manipulate a computing device, and can include an output for allowing the computing device to present information and/or data, indicate the effects of the user's manipulation, etc. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

As referred to herein, the terms “physical sensor” or “sensor” generally refer to a device that detects and responds to an input from a physical environment. The physical sensor can convert the detected input into a signal that is representative of the detected input. For example, a physical sensor can be used to monitor and acquire a subject's (or person's) vital signs, movement, temperature, sleep patterns, activity level, electrocardiogram (ECG) readings, and the like. Physical sensors can be implemented to include electrical, mechanical, optical, chemical, and/or biological sensing mechanisms. Example physical sensors include, but are not limited to, polysomnography equipment, various wearable devices, accelerometers, motion sensors, fitness trackers, medical devices. A physical sensor can acquired objective health data about the subject that is measurable and quantifiable data about the subject's health. Sensors may also include devices that are not connected to the body for example radar, camera, or other off-body sensor types.

As referred to herein, the term “subject” generally refers a person, such as a patient, receiving healthcare treatment or who is being evaluated, diagnosed, or assessed for healthcare treatment, planning, and/or reporting.

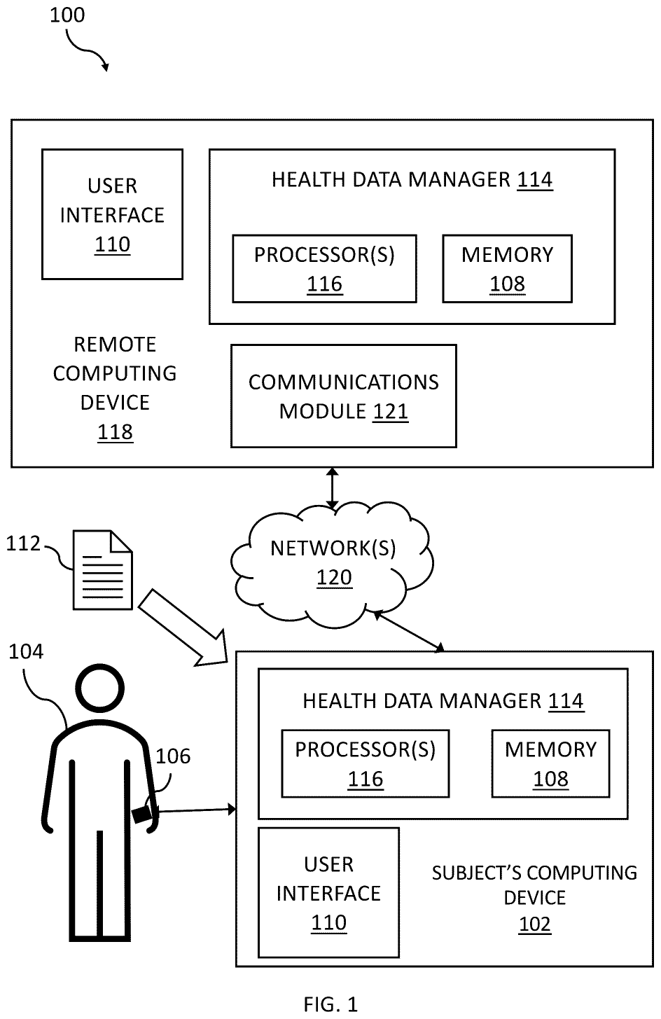

Systems, devices, and methods disclosed herein can be utilized for analyzing objective health data and subject-conveyed health information for health information reporting, treatment planning, and diagnoses. FIG. 1 illustrates a block diagram of a system 100 for analyzing objective health data and subject-conveyed health information for health information reporting, treatment planning, and diagnoses in accordance with embodiments of the present disclosures. Referring to FIG. 1, the system 100 includes a computing device 102 that may be carried by a subject 104 or otherwise associated with the subject 104. For example, the computing device 102 may be a smartphone, tablet computer, or other device that is operable to communicate with a physical sensor 106 being operably worn by the subject 104 or otherwise configured to acquire objective health data of the subject 104. For example, the physical sensor 106 can detect the subject's (or person's) vital signs, movement, temperature, and/or the like, and communicate the detected health data to the subject's computing device 102. This objective health data may be suitably stored in memory 108 of the computing device 102. It is noted that although only one physical sensor 106 is shown in FIG. 1, it should be understood that more than one physical sensor of the same or different types may be operationally worn, attached to, or placed near the subject 104 for acquiring the objective health data.

The system 100 may receive various other information about the subject's 104 health. In accordance with embodiments, the subject 104 may render an opinion about the subject's health and convey the opinion directly or indirectly to the computing device 102. For example, the subject 104 may interact with a user interface 110 of the computing device 102 to enter or convey the subject's opinion about a perceived physical characteristic or perceived experience about the subject's health. Such information may be suitably entered or conveyed as data 112 and entered into the computing device 102 via the user interface 110. Example the subject 104 may enter the subject's perceived total sleep time into the computing device 102 via the user interface 110. The information may be received at different times and be time stamped as the time of entry or some indicia of when the subject experienced the health condition. The information may be received and stored in memory 108.

The computing device 102 includes a health data manager 114 configured to receive a subject's objective health data and information about the subject's health that was conveyed by the subject. Further, the health data manager 114 can operate the user interface manager 110 for displaying or otherwise presenting the objective health data and subject's other health information to an operator, such as the subject 104. In examples, the health data and information may be suitably displayed as a numbers representative of the information, graphs, images, and/or the like.

The health data manager 114 may include any suitable hardware, software, and/or firmware for implementing the functionalities described herein. For example, the health data manager 114 includes the memory 108 and one or more processors 116 for implementing the described functionalities.

The system 100 may also include a remote computing device 118 that is communicatively connected to the subject's computing device 102 via one or more communications networks 120. The remote computing device 118 may be a server, laptop computer, tablet computer, or other computing device operated by a healthcare practitioner. Further, the remote computing device 118 may include a health data manager 114 operable to implement the functionalities described herein. In this example, the health data manager 114 include memory 108 and processor(s) 116. In embodiments, the subject's computing device 102 may collect and store collected objective health data and other health information of the subject 104, and subsequently communicate the objective health data and health information of the subject 104 to the remote computing device 118 via the network(s) 120. The remote computing device 118 can include a communication module 121 for communicating with the network(s) 120.

At the remote computing device 118, the health data manager 114 can analyze, the objective health data and the health information of the subject 104. Further, the health data manager 114 can determine a treatment plan, diagnosis, and/or health information based on the analysis in accordance with embodiments of the present disclosure. Further, the health data manager 114 can use the user interface 110 of the remote computing device 118 to present the treatment plan, diagnosis, and/or health information to the healthcare practitioner.

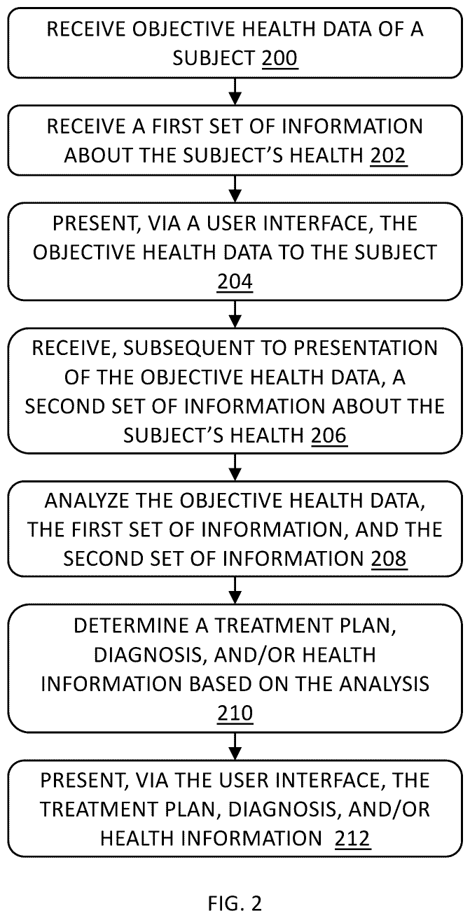

FIG. 2 illustrates a method of analyzing objective health data and subject-conveyed health information for health information reporting, treatment planning, and diagnoses in accordance with embodiments of the present disclosure. The method is described by example as being implemented by the subject's computing device 102 shown in FIG. 1, although it should be understood that the method may be implemented by the remote computing device 118 or another suitable computing device. The computing device 102 and the remote computing device may be suitably configured to receive objective health data of the subject 104 and health information conveyed by the subject 104.

Referring to FIG. 2, the method includes receiving 200 objective health data of a subject. For example, the physical sensor 106 may acquire objective sleep data about the subject 104. In an example, the physical sensor 106 may be worn on the ankle or wrist of the subject 104 and operable to acquire data for use in estimating one or more sleep metrics such as, but not limited to, total sleep time, sleep onset latency, wake after sleep onset, sleep efficiency, and/or the like. The health data manager 114 can use these metrics for predicting autonomic and cortical arousals associated with leg movements during the subject's sleep. The acquired objective health data may be suitably stored in memory 108 of the subject's computing device 102.

The method of FIG. 2 also includes receiving 202 a first set of information about the subject's health. Continuing the aforementioned example, the subject 104 may interact with the user interface 110 of the subject's computing device 102 to enter information about the subject's health. For example, the entered information may indicate amount of total sleep time of the subject 104, which is the total amount of time that the subject 104 believes or perceived she or he slept within a time period (e.g., at regular night time sleep). This information may be entered prior to the subject's knowledge of the actual measured total sleep time within the same time period or another measure of sleep. The entered information may be stored in memory 108 of the subject's computing device 102.

The method of FIG. 2 includes presenting 204, via a user interface, the objective health data to the subject. Continuing the aforementioned example, the health data manager 114 may utilize the user interface 110 to present (e.g., display) some or all of the acquired objective health data of the subject 104. For example, the user interface 110 can disclosed the stored total sleep time, sleep onset latency, wake after sleep onset, and/or sleep efficiency. This objective health data may correspond to the same sleep time as the subject 104 entered the information of block 202. Also, this objective health data may be presented to the subject 104 subsequent to the subject 104 entering the information of block 202. The presentation of this data is later so that the subject 104 is without foreknowledge of the actual measured sleep information.

The method of FIG. 2 includes receiving 206, subsequent to presentation of the objective health data, a second set of information about the subject's health. Continuing the aforementioned example, the subject 104 may interact with the user interface 110 of the subject's computing device 102 to enter another set of information about the subject's health. This information may be enter subsequent to entry of the information at block 202 and subsequent to presentation of the objective health data at block 204. For example, the entered second set of information may indicate amount of total sleep time of the subject 104, which is the total amount of time that the subject 104 believes or perceived she or he slept within another time period (e.g., at regular nighttime sleep). For example, this may be the perceived amount of sleep for a later night or multiple later nights. The entered second set of information may be stored in memory 108 of the subject's computing device 102.

The method of FIG. 2 includes analyzing 208 the objective health data, the first set of information, and the second set of information. Continuing the aforementioned example, analysis may include determining a variance, mean, and/or the like of the objective health data, the information received at block 202, and the information received at block 206.

The method of FIG. 2 includes determining 210 a treatment plan, diagnosis, and/or health information based on the analysis. Continuing the aforementioned example, the health data manager 114 may determine variance and/or mean of the objective health data, the information received at block 202, and the information received at block 206. In another example, the objective health data can be data acquired about a predetermined health characteristic of the subject 104 within a predetermined period of time, and the initial set of information obtained at block 202 is associated with the subject's conveyance of opinion about the predetermined health characteristic of the subject within the predetermined period of time. Continuing this example, the health data manager 114 can compare the data about the predetermined health characteristic with the subject's conveyed opinion about the predetermined health characteristic; and present, via the user interface 110, the comparison of the data about the predetermined health characteristic with the subject's conveyed opinion about the predetermined health characteristic.

In another example, the objective health data can include data acquired about a predetermined health characteristic of the subject within a plurality of different periods of time; and the initial set of information obtained at block 202 can be about the subject's health associated with the subject's conveyance of opinion about the predetermined health characteristic of the subject within the plurality of different periods of time. The health data manager 114 can present, via the user interface 110, the data about the predetermined health characteristic and the subject's conveyed opinion about the predetermined health characteristic.

The method of FIG. 2 includes presenting 212, via the user interface, the treatment plan, diagnosis, and/or health information. Continuing the aforementioned example, the health data manager 114 may utilize the user interface 110 to present determined variance and/or mean of the objective health data, the information received at block 202, and the information received at block 206.

In embodiments, the systems, devices, and methods disclosed herein can be used to identify discrepancies between self-reported and sensor-based sleep metrics, analyze the discrepancies, and present treatment plan, diagnosis, and/or health information based on the identified discrepancies. Sleep wake state discrepancy (SWSD) is commonly experienced by individuals with insomnia and has been noted in those with other sleep disorders, as well as in the general population. SWSD is characterized by an overestimation of sleep onset latency (SOL) and waking after sleep onset (WASO) and underestimation of total sleep time (TST) relative to objective measures of sleep. Individuals with SWSD report poor sleep quality and excessive daytime sleepiness; however, despite claims of minimal or no sleep over extended periods of time, PSG results reveal near-normal sleep patterns. In order to address the discrepancy, studies have examined the impact of providing feedback about the discrepancy between self-reported and sensor-based sleep data. It should be noted that although sleep is referred to herein in examples, the same or similar techniques may be applied to other physical states or behaviors.

It is noted that wearable sensors/devices and sleep diaries are often used as part of a multi-method sleep assessment approach in research. In fact, it is recommended that wearable data should be interpreted in conjunction with sleep diary data. This allows for adjustment of factors that may not be detectable by a wearable device (e.g., sitting very still in bed watching TV without the intention to sleep may be incorrectly interpreted as sleep time by a wearable device; the sleep diary allows the participant to indicate their intended time in bed which can then be used to adjust the rest interval). However, comparing wearable device and sleep diary data across sometimes hundreds of nights of data is a laborious task that requires researchers to make assumptions about a participant's data without the participant's input, long after the data was collected. Instead, allowing participants to make these adjustments themselves would likely increase efficiency and improve accuracy without adding a great deal of participant burden.

Systems, devices, and method disclosed herein can streamline and integrate feedback from sleep diaries and sensor-based wearable devices. In experiments, a sleep diary is combined with sensor-based assessment of sleep by providing users with their wearable device data on a daily basis when they complete their sleep diary. This allows users to contextualize their response while retaining their autonomy to be the final arbiter of the assessment. From a signal processing perspective, the combination of data from two sensors is mathematically guaranteed to provide a better estimate of the underlying signal assuming that their noise is uncorrelated. In this application, the sleep diary and the wearable device can be considered two sensors with uncorrelated sources of noise. Given this, their combination can improve the estimate of the underlying metrics.

In experiments, the effects of providing sensor-based device data, concurrently with the completion of a sleep diary on a daily basis, were examined to improve sleep assessment for applications in research and therapeutic development. In the experimental sample of otherwise healthy individuals (i.e., no known sleep pathology), it was hypothesized that the integrated sleep diary as compared to a control diary (with no sensor-based feedback), would: 1) reduce variance in self-reported TST and SOL; 2) have no effect on the mean of self-reported TST and SOL 3) improve agreement of self-reported TST and SOL with the sensor-based sleep parameters; and 4) improve confidence in the self-report. In addition, exploratory analyses were conducted to measure the effect of the integrated sleep diary on sensor-based sleep parameters, self-reported sleep quality ratings, and the identification of individuals with SWSD.

In one study, after collection of baseline data, participants were randomly assigned to begin in one of two sequences: (A) completion of the control diary for one week followed by a one-week washout period, and then completion of the integrated diary for one week, or (B) completion of the integrated diary for one week followed by a one-week washout period, and then completion of the control diary for one week. Diary entries were completed upon waking to assess self-reported TST and SOL, confidence in each sleep parameter (or generally a “physical parameter”), and sleep quality. Sensor-based device data and sleep diary entries were not collected during the washout period.

Participants in each condition completed the same daily sleep diary questions on a proprietary application installed on their mobile device. On the first screen, participants in both conditions answered the following three questions about their sleep: 1) How long did you sleep last night? 2) How long did it take you to fall asleep last night? and 3) How well did you sleep last night? Responses for the sleep quality questions utilized a 5-point Likert scale (1-5, with 5 indicating excellent sleep). On the second screen participants in both conditions were asked:, 1) “How confident are you in your rating of SOL?” and 2) “How confident are you in your rating of TST (using a 5-point Likert scale 1-5, with 5 indicating the highest confidence)?”

Identification of SWSD was based on the discrepancy between the sleep diary entry for SOL in Sequence A, Period 1 and the SOL sensor-based value for the same Sequence and Period. In Sequence A, participants began with the control diary. Participants with values >2 SD above the mean (M=29.9 (24)) were identified as showing evidence of SWSD if more than half of their study nights met these criteria. 3 participants were identified who met this criterion.

The integrated sleep diary is engineered to integrate objective sleep data from the wearable device (i.e., a FITBIT Inspire 2) into the mobile sleep diary application. Participants saw a declarative statement that reported what data the wearable device collected the night before the morning sleep diary entry, for example, “Your wearable device reports that you slept 7 hours and 30 minutes last night” and “Your device says it took you 30 minutes to fall asleep last night.” This information was displayed above the sleep diary questions, “How long did you sleep last night?” and “How long did it take you to fall asleep last night?” The control diary did not provide wearable device data in conjunction with the sleep diary question in the mobile application.

Analyses were conducted using R version 4.3.0, utilizing the following packages: lubridate, mgcv, brms, SimplyAgree, bayestestR, and see. Python version 3.10.9 was also used, with the inclusion of the Pandas, NumPy, and Matplotlib libraries. Differences by condition (number of study nights completed, proportion of diary entries matching the sensor-based value) were tested using t-tests or X2 tests as appropriate. Bayesian distributional analysis was used to assess the shape and change of the TST and SOL parameters as a function of using the integrated diary (Makowski et al., 2019). Distributional models are a class of regression models that allow for modeling not only the mean but also the variance and higher moments of the response distribution as a function of covariates. For the outcome of this analysis, we report the median as an index of centrality with the associated interquartile range (IQR), the probability of direction (pd) as the percent certainty associated with the most likely direction of the effect (positive or negative), 95% credible intervals (CI) characterize the span of uncertainty associated with the estimation, and the percentage in the region of practical equivalence (ROPE).

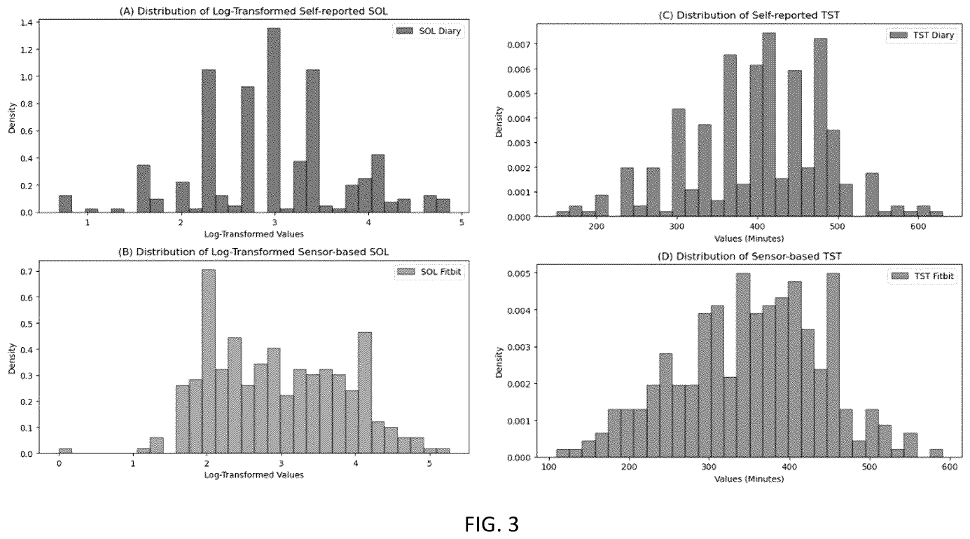

FIG. 3 are graphs showing data distributions of self-reported data for sleep parameters. Given the distributions of the data shown in FIG. 3, non-parametric Bland-Altman analysis was conducted to examine the limits of agreement and bias between self-reported and sensor-based sleep parameters. The thresholds for the minimum allowable differences between the diary and wearable device (limits of agreements; LOA) were set to 30 minutes for SOL and 60 minutes for TST. Finally, binomial proportions testing was conducted to determine the number of data points within those bounds at 5 minute increments from 5 minutes to 60 minutes for SOL and TST.

Participants (N=24, M age=22.36 [SD=3.9], range 19-40; 50% female) reported no known history of cardiovascular, respiratory, or sleep-related disorders. All participants completed the full 3-week study. In the control diary condition the average number of nights completed was 6.75(1.89) and in the integrated diary condition it was 5.25(1.98) the difference between conditions was not significant t (22)=1.90, p>0.05. Table 1 below reports the median, and IQRs for the sleep parameters (TST, SOL) for each sequence and period.

TABLE 1
Self-reported and sensor-derived descriptive statistics for sleep parameter
variables as a function of study design by sequence and period

| | | Me- | | | Me- | |
|---|---|---|---|---|---|---|
| | Period 1 | dian | IQR | Period 2 | dian | IQR |
| Sequence | TST Diary | 440.0 | 116.25 | TST Diary | 390.0 | 128.75 |
| A | TST Fitbit | 360.5 | 130.75 | TST Fitbit | 344.5 | 126.0 |
| | SOL Diary | 15.5 | 20.00 | SOL Diary | 20.0 | 15.00 |
| | SOL Fitbit | 18.5 | 25.0 | SOL Fitbit | 18.0 | 42.25 |
| Sequence | TST Diary | 390.0 | 87.50 | TST Diary | 420.0 | 116.25 |
| B | TST Fitbit | 360.0 | 98.0 | TST Fitbit | 375.5 | 129.5 |
| | SOL Diary | 20.0 | 29.25 | SOL Diary | 18.0 | 15.00 |
| | SOL Fitbit | 17.0 | 20.75 | SOL Fitbit | 15.5 | 42.25 |

Note:
IQR = Interquartile Range;
TST = total sleep time;
SOL = sleep onset latency

Control diary entries were the same as the sensor-based sleep parameter a total of 7 times (2% of control diary entries). Integrated diary entries were the same as the sensor-based sleep parameter 23 times (9% of the integrated diary entries). A Chi-Square Goodness of Fit Test was performed to determine whether the proportion of diary entries with the same value as the sensor-based data was equal between the control and integrated diary conditions. The proportions differed by 21%, 2(1, x 24)=62.806, p<0.0001.

The assumptions for Bland-Altman analysis includes normal distribution of the measurement differences and proportional bias. To test whether our data met these assumptions we conducted the Shapiro Wilk Test and the Test for Linear Bias on the SOL and TST distributions. For SOL and TST, the residuals were not normally distributed as indicated by failure of the Shapiro Wilk Test p>0.05. The Test for Linear Bias (p>0.05) revealed lack of proportional bias for SOL and TST. The proportional bias was clearly violated in the case of SOL and for the sake of comparability between analyses, a non-parametric method was used by including a slope coefficient and intercept in the regression method of calculating LOA for both SOL and TST.

The assumptions for linear regression include normality of residuals and homogeneity of variance. To test these assumptions, the Shapiro Wilk Test and Breusch Pagan Test were used. For SOL and TST, the residuals were not normally distributed as indicated by failure of the Shapiro Wilk Test p>0.05 and a significant Breusch-Pagan Test p<.05 indicated heteroskedasticity in the data variance for both SOL and TST. The interpretation of these findings demonstrates that overall the sleep parameter (TST, SOL) data violated the assumptions for linear regression analyses which necessitated the use of a Bayesian distributional approach.

Distributions for self-reported and sensor-based TST and SOL are shown in FIG. 3. One outlier (>3 SD above the mean) in the SOL sensor-based distribution (not shown) was removed from analysis due to an impossible/unlikely value. The self-reported SOL variable was log transformed to attain residual normality. For presentation purposes logged values were exponentiated for interpretation when presented in tabular format.

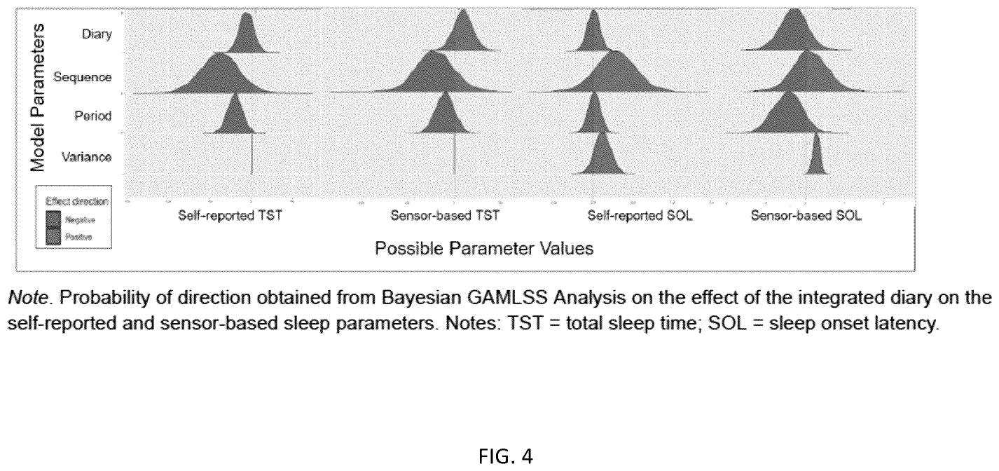

Findings from the Bayesian distributional analysis to assess the shape and centrality of the distribution for self-reported TST parameter as a function of the integrated diary condition are summarized in FIG. 4 and Table 2 below. As can be seen, the analysis indicated that the diary decreased self-reported TST. Participants who began with Sequence B (integrated diary first) showed shorter TST than Sequence A. Participants reported shorter TST when comparing Period 2 to Period 1. The integrated diary decreased the variance in TST reporting.

TABLE 2
Results of Bayesian Distributional Analysis
on the Effect of the Integrated Diary on Self-
Reported and Sensor-Based Sleep Parameters

| | | Direc- | | HDI_{low}- | |
|---|---|---|---|---|---|
| | Estimate | tion | pd | HDI_{high} | ROPE |
| Self- | | | | | |
| reported TST | | | | | |
| Integrated | −6.16 | − | 76.17% | −23.58-10.71 | 58.48 |
| Diary | | | | | |
| Sequence | −37.68 | − | 94.90% | −83.46-7.33 | 7.85 |
| Period | −20.48 | − | 98.75% | −38.09-−2.41 | 10.65 |
| | −0.31 | − | 99.95% | −0.51-−0.14 | 100 |
| Self- | | | | | |
| reported SOL | | | | | |
| Integrated | 1.00 | | 50.55% | −0.16-0.14 | 70.62 |
| Diary | | | | | |
| Sequence | 1.33 | + | 87.12% | −0.23-0.76 | 13.30 |
| Penod | 1.00 | | 54.73% | −0.13-0.16 | 70.38 |
| Variance | 1.12 | + | 88.20% | −0.08-0.31 | 34.70 |
| Sensor- | | | | | |
| based TST | | | | | |
| Integrated | 8.90 | + | 81.95% | −10.79-28.18 | 46.10 |
| Diary | | | | | |
| Sequence | −20.01 | − | 84.23% | −57.65-19.86 | 20.28 |
| Period | −10.18 | − | 84.12% | −29.59-9.29 | 43.30 |
| Variance | −0.10 | − | 100% | −0.28-0.07 | 100 |
| Sensor- | | | | | |
| based SOL | | | | | |
| Integrated | −0.74 | − | 81.35% | −0.97-0.39 | 45.88 |
| Diary | | | | | |
| Sequence | 1.10 | | 59.98% | −0.80-1.00 | 49.28 |
| Period | 0.63 | − | 89.50% | −3.17-0.28 | 32.42 |
| Variance | 1.32 | + | 99.95% | 0.09-0.11 | 36.47 |

For self-reported SOL, the analysis indicated that the integrated diary did not alter centrality measures of SOL. An effect for sequence but not period can be seen. Unexpectedly, the integrated diary increased the variance in SOL reporting (See FIG. 4 and Table 2).

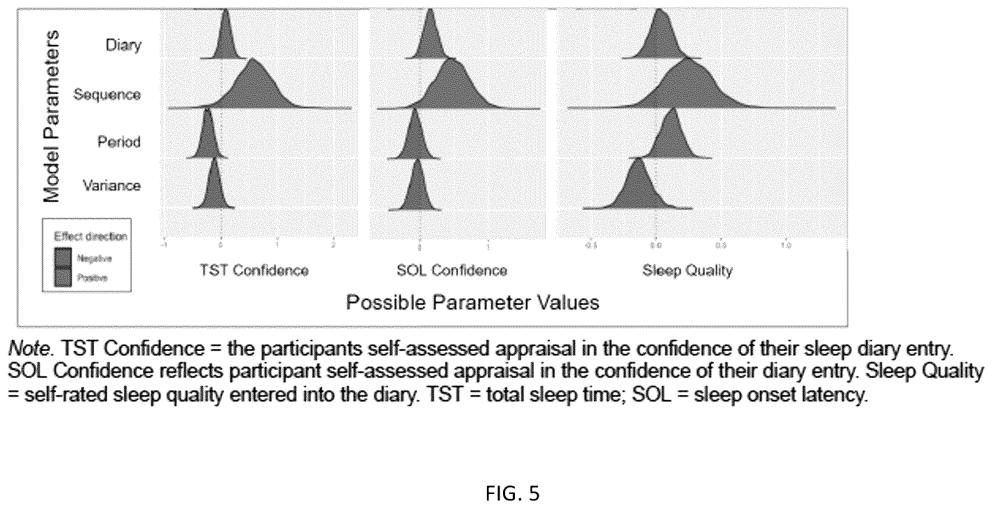

When participants completed their diary entry every morning they rated confidence in their assessment of TST and SOL, and for sleep quality (SQ) on a likert scale (1-5; 5 indicating the best quality). Table 3 below shows the median, mean, SD, and interquartile range for TST, SOL, and SQ as a function of sequence and period. FIG. 5 and Table 4 below shows results of the GAMLSS analysis that indicated the integrated diary increased confidence in the TST diary entry. Participants who began with Sequence B appraised their confidence in TST more than Sequence A. Participants' confidence in their TST diary entries were less when comparing Period 2 to Period 1. Finally, compared to the control diary, confidence appraisals for TST for the integrated diary had less variance.

TABLE 3
Confidence Ratings for Sleep Parameters

| | Peri- | Me- | | | | Peri- | Me- | | | |
|---|---|---|---|---|---|---|---|---|---|---|
| | od 1 | dian | IQR | M | SD | od 2 | dian | IQR | M | SD |
| A | TST | 3.0 | 2.0 | 3.1 | 1.2 | TST | 4.0 | 1.0 | 3.48 | 0.93 |
| | SOL | 3.0 | 2.0 | 3.0 | 1.1 | SOL | 3.0 | 1.0 | 3.48 | 0.84 |
| | SQ | 3.0 | 1.0 | 2.7 | 0.8 | SQ | 3.0 | 1.0 | 3.06 | 0.72 |
| B | TST | 4.0 | 1.0 | 3.8 | 0.8 | TST | 3.0 | 2.0 | 3.48 | 0.93 |
| | SOL | 4.0 | 1.0 | 3.7 | 0.8 | SOL | 3.0 | 2.0 | 3.48 | 0.84 |
| | SQ | 3.0 | 0.0 | 3.0 | 0.7 | SQ | 3.0 | 1.0 | 3.06 | 0.72 |

Notes:
A = Sequence A;
B = Sequence B;
total sleep time = TST;
sleep onset latency = SOL;
and sleep quality = SQ;
interquartile range = IQR;
mean = M;
standard deviation = SD

TABLE 4
Bayesian GAMLSS Analysis for the Effect of the Integrated
Diary on daily confidence ratings of diary entry
for TST, SOL, and appraisal of sleep quality

| | | Direc- | | HDI_{low}- | |
|---|---|---|---|---|---|
| | Estimate | tion | pd | HDI_{high} | ROPE |
| Confidence TST | | | | | |
| Integrated Diary | 0.08 | + | 82.70% | −0.30-0.25 | 59.33 |
| Sequenos | 0.58 | + | 95.58% | −0.32-1.20 | 5.25 |
| Period | −0.23 | − | 99.22% | −0.41-0.05 | 8.88 |
| Variance | −0.12 | − | 89.98% | −0.29-0.06 | 44.15 |
| Confidence SOL | | | | | |
| Integrated Diary | 0.15 | + | 93.90% | −0.04-0.34 | 26.07 |
| Sequence | 0.47 | + | 96.88% | −0.03-0.95 | 5.65 |
| Period | −0.07 | − | 77.33% | −0.26-0.11 | 58.05 |
| Variance | −0.04 | − | 65.77% | −0.22-0.14 | 68.17 |
| Sleep Quality | | | | | |
| Integrated Diary | 0.04 | + | 67.07% | −0.12-0.19 | 62.32 |
| Sequence | 0.25 | + | 89.28% | −0.11-0.62 | 15.17 |
| Period | 0.11 | − | 91.30% | −0.03-0.26 | 34.35 |
| Variance | −0.14 | − | 92.17% | −0.31-0.05 | 24.27 |

The integrated diary increased confidence in the SOL diary entry. Participants who began with Sequence B appraised their confidence in SOL more than Sequence A. Participants' confidence in their SOL diary entries were lower when comparing Period 2 to Period 1. Finally, compared to the control diary, confidence appraisals for SOL for the integrated diary were less variable. These results are presented in FIG. 5 and Table 4.

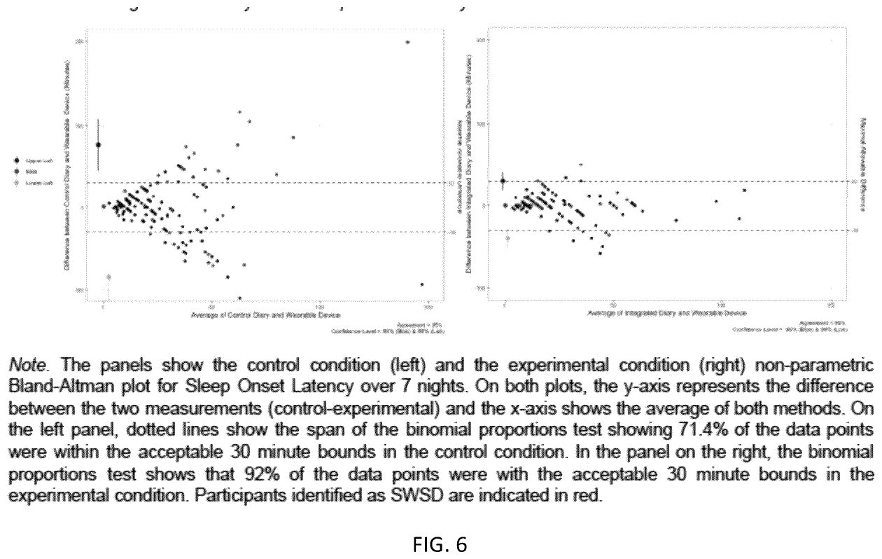

The non-parametric Bland Altman analyses were conducted to determine the agreement between the sensor-based and self-reported estimates of SOL and TST. As can be seen on the left panel in FIG. 6, for SOL and the control diary, the quantile limits of agreement (displayed on the plot) indicate minimal bias (−2.73 minutes) but relatively wide bounds (Upper 74.33 mins, lower −2.73 mins). On the panel on the right, the integrated diary condition, the quantile limits of agreement indicate minimal bias (0.48 mins) and narrower bounds compared to the control condition (Upper 35.44, lower −38.97). As can be seen, SWSD participants, indicated in dark orange, in the integrated diary show evidence of changing their perception of sleep to be closer in agreement with sensor-based data. These findings suggest that the integrated diary has better agreement (lower variance) between control and integrated diary conditions.

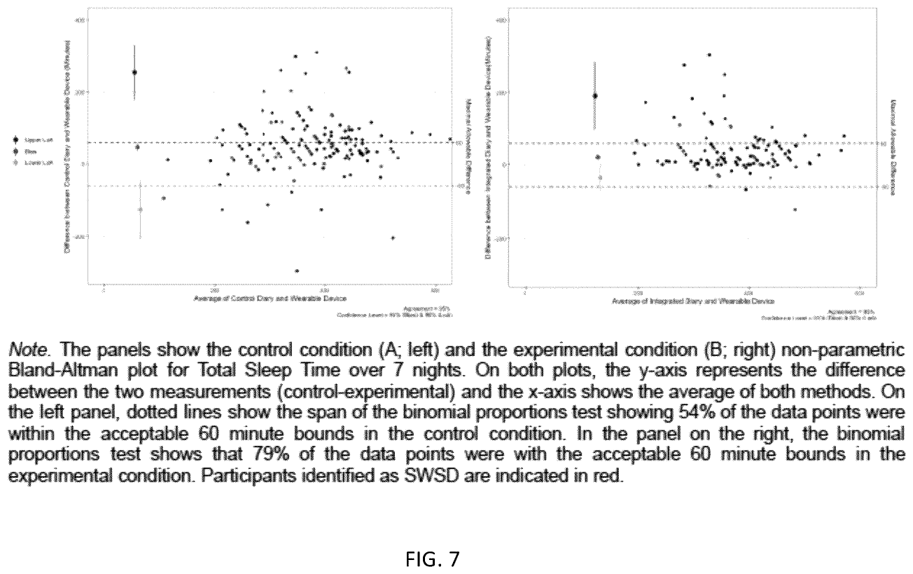

As can be seen on the left panel in FIG. 7, the quantile limits of agreement (displayed on the plot) indicate positive and asymmetric bias (47.74 mins) and wide bounds (Upper 261.6 mins, lower −127 mins). On the panel on the right, the integrated diary, the quantile limits of agreement indicate less bias (22.1 mins) and more narrow bounds (Upper 231.5, lower −51.55 mins) compared to the control diary. As can be seen, SWSD participants using the integrated diary show evidence of changing their perception of sleep to be closer in agreement with the sensor-based data as they switch from the control to the integrated diary. These findings suggest that the integrated diary has substantially better agreement (lower variance) with sensor-based data and may impact sleep related perception.

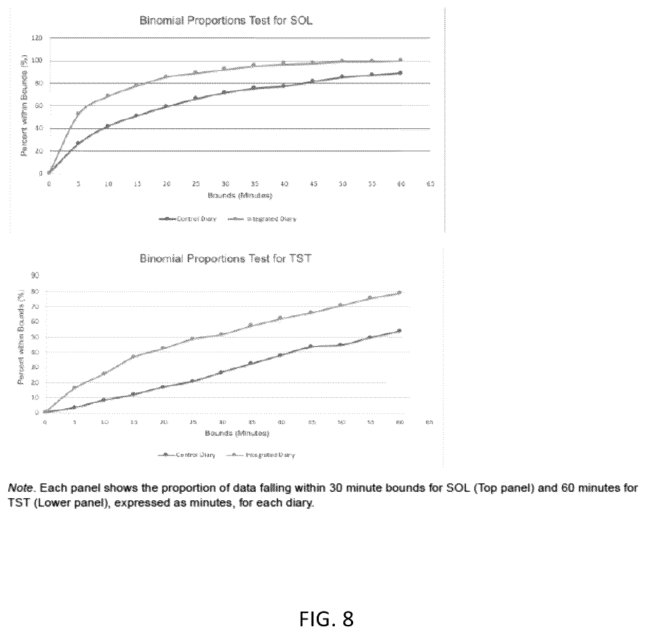

Exploratory analyses was conducted to understand the proportion of data points within 5 minute incremental bounds from 5 minutes to 60 minutes for TST and for SOL and those results are shown in FIG. 8. As can be seen at every incremental value the integrated diary includes a larger proposition of data points than the control diary.

As can be seen in FIG. 4 and Table 2, the integrated diary increased sensor-based TST. Participants who began with Sequence B showed less TST than Sequence A and participants reported less TST when comparing Period 2 to Period 1. The integrated diary decreased the variance in TST reporting.

The integrated diary had no effect on sensor-based SOL. Participants who began with Sequence B showed a slightly longer SOL than Sequence A and no effect when comparing Period 2 to Period 1. Consistent with the same finding for the SOL diary entry, the integrated diary increased the variance in objective SOL.

As can be seen, the results of the distributional analysis shown in FIG. 5 and Table 4 indicate the integrated diary increased sleep quality appraisal. Participants who began with Sequence B appraised their sleep quality better than Sequence A. Comparing Period 2 to Period 1, participants rated their sleep quality better. Finally, compared to the control diary, confidence appraisals for SOL for the integrated diary had less variance.

In another study, paradoxical insomnia was investigated. Paradoxical insomnia, also known as sleep-wake state discrepancy (SWSD) is a common phenomenon in insomnia where individuals characteristically report less sleep and more wakefulness than what is measured by polysomnography (PSG). Evidence suggests that individuals with SWSD are sensitive to cortical arousal and often sense that they are awake during (objectively measured) sleep raising the potential that SWSD may be characterized in part by heightened interoception. It is believed a sleep diary that supplements daily diary entries with objectively derived sleep data may have therapeutic effects in SWSD. It is hypothesized that the SWSD phenotype is hyper-aware of cortical arousal such that upon waking, periods of enhanced arousal are perceived as wakefulness, despite the objective definition of being asleep, generating the discrepancy between self-report and objective measures, giving rise to the SWSD. Within the context of neurocognitive and hyperarousal/sustained wakefulness theories of insomnia for SWSD specifically, reappraising sleep due to daily awareness of discrepancy could initiate therapeutic changes in sleep-perception and reduce sleep-related distress thereby plausibly reverse or diffuse negative sleep bias, improve SQ and duration and provide support for a precision medicine model for SWSD intervention.

Preliminary data was obtained with a study of participants (n=24, 288 nights) who wore a wearable device during the nightly sleep period (To improve estimation of sleep onset latency (SOL) and completed a standard diary (SD) and an integrated sleep diary (ISD), in a cross-over study. The ISD, displayed nightly device-derived sleep metrics when daily diary entries were completed which were not displayed in the SD. There was a trend suggesting the ISD increased sleep quality appraisal, device-measured total sleep time, and reduced sleep latency. Unexpectedly, the ISD identified SWSD in 3 individuals who, compared to the SD, showed a dramatic reduction in SWSD, suggesting the provision of the device data improved perception. A manuscript reporting these effects has been reviewed and is under revision. In related work, a machine learning algorithm was developed and validated for an ankle-worn wearable device, referred to as RESTEAZE™, that estimates standard sleep metrics including TST, SOL, and WASO validated against PSG and predicts autonomic and cortical arousals associated with leg movements during sleep (AUC of 0.92).

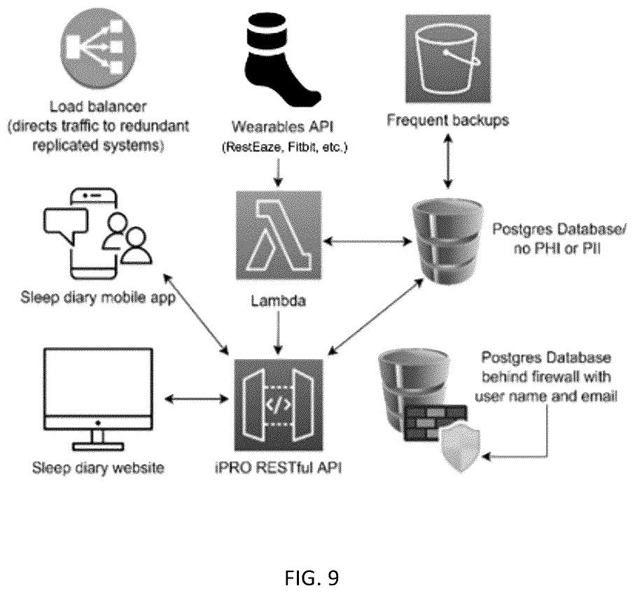

In accordance with embodiments, FIG. 9 illustrates a block diagram of another system for analyzing objective health data and subject-conveyed health information for health information reporting, treatment planning, and diagnoses in accordance with embodiments of the present disclosures. Referring to FIG. 9, user interface endpoint (i.e., the website and mobile application) are connected to a webservice (referred to as RESTful web service) that provides interaction with a main database. The RESTful web service can be secured by a suitable token. These may be hosted on a Windows server elastic compute (EC2) for example. Several middle layer processes can be deployed on AWS Lambda to interact with the wearable devices and the main database instead of using a separate application server for cost savings. The main database is a Postgres database that contains no PHI/PII. It houses the data using a machine generated identifier for users.

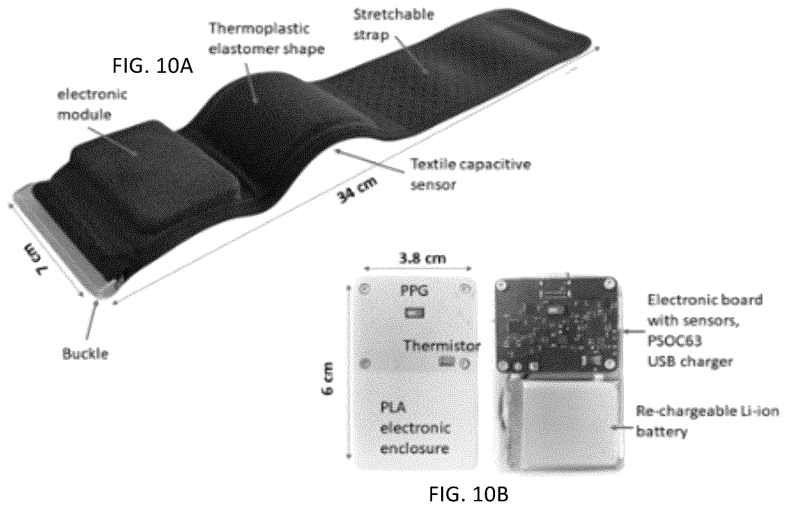

The ISD-Sleep Diary is a platform technology for implementing the system of FIG. 9 that comprises a wearable device worn on each ankle, a web application-based sleep diary (that shows wearable device data), a set of business rules, video-based instructions, and back end data aggregation/management system. The system can log sleep data from the device and diary, manage communications for alerts (to remind patients to complete the sleep diary), and monitor for compliance on HIPAA-compliant AWS instances to ensure data security/privacy. It is designed with redundancy to ensure >99% uptime in the face of server outages. The wearable device can be the FITBIT™ watch, the APPLE watch, or the OURA RING™ into the ISD ecosystem. This ankle-worn sleep-dedicated wearable device includes a proprietary sensor suite that analyzes leg movements during sleep and standard sleep metrics (see FIGS. 10A-10C).

FIG. 10A is an image showing various soft goods for the device including breathable washable neoprene, a flexible plastic mold of the shin and a pocket for the electronics module. FIG. 10B is an image showing the device being worn on a subject's ankle. FIG. 10C is an image showing contents of the electronics module including the 3-LED photoplethysmography (PPG) sensor, thermistor, battery, charge circuitry, and processor.

Based on anatomical and functional connections between the brain and spinal cord, and validated with PSG, the analytics classify leg movements into those associated with micro-cortical brain arousal. An integrated electronics module captures heart rate, heart rate variability, blood pressure, and body temperature to detect autonomic arousals (transient, significant elevations in heart rate and blood pressure). To improve the precision of sleep onset latency, specific instructions were provided to participants to optimize measurement. Patients are instructed to put the device on right at bedtime and to remove it when they wake up for the day. These instructions enable more precise measurement of both the subjective estimation (i.e. users are advised to note the time that they put on the device) and objective estimation (by limiting the timeframe over which sleep can be detected). The sleep diary component of the system was designed to preclude copy/paste actions and remind users that sleep devices are imperfect. These manipulations help ensure that users use the device data to contextualize their responses rather than directly replicating them.

The system shown in FIG. 9 can be used to advance the science of the interrelationships between insomnia, SWSD, and physiological arousal to gain insight into potential mechanistic explanations for discrepant sleep perception and therapeutic response to ISD. It differs from the other studies that have integrated wearable device data in aggregate in that the intervention shows device data, daily, to users while they complete questions related to the appraisal of their sleep in a sleep diary with the reminder that devices are imperfect measures. Despite advances in sleep science medicine, insomnia remains endemic in our society and up to 40% of individuals seeking treatment fail to respond to conventional treatments. Systematic attention to objective sleep data alongside questions related to the user's first-person appraisal of their sleep may offer an innovative, simple, and timely assessment of sleep that offers therapeutic benefits. The ISD identifies cases of sleep-wake state discrepancy in an approach that is more feasible than polysomnography and potentially of great utility to sleep clinics with lengthy wait lists.

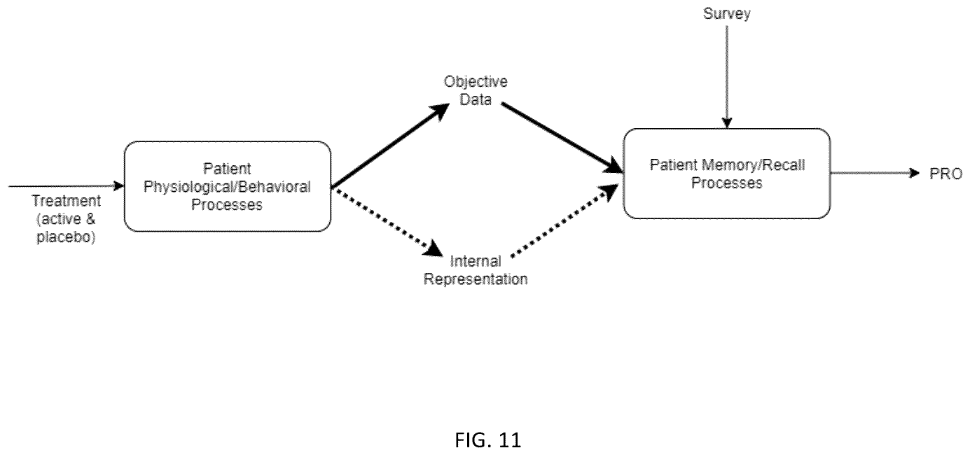

By use of the systems, devices, and methods disclosed herein, the internal memory/recall processes of the patient are augmented by providing objective reference data to the patient to help contextualize their responses. FIG. 11 illustrates a schematic of an example system for assisting subjects to contextualize their responses in accordance with embodiments of the presented disclosure. Referring to FIG. 11, a representation of objective data are provided to the patient at the time of survey completion that can be referenced in order allow the patient to generate a more accurate response. This method has been successfully applied in physical activity monitoring wherein GPS/accelerometry data were provided to runners to improve their recall of physical activity. Further, a similar approach has been used for depression research where an improved separation between treatment and placebo was demonstrated when patients were asked how they felt in reference to listening to a vocal recording of themselves at baseline. Model of improved PROs. In the schematic of FIG. 11, the improved PRO is presented wherein, objective data that captures similar physiologic/behavioral changes is presented to the patient at the time of survey completion that can augment patient memory/recall process and more accurately represent treatment effect. In summary, an advantageous example feature of methods and systems disclosed herein is to provide objective data cues to patients at the time of survey to generate more accurate PROs.

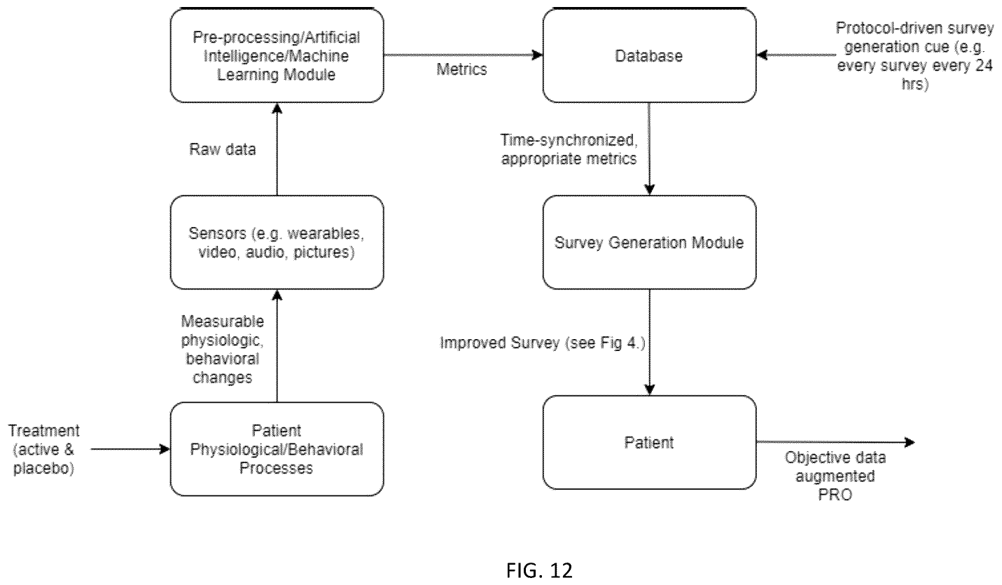

FIG. 12 illustrates a high-level schematic of a system in accordance with embodiments of the present disclosure. This system is embodied by the “Objective Data” section of FIG. 12, examples of which are provided in Table 5 below. In the system of FIG. 12, treatment is provided to the patient that imparts physiologic and/or behavioral changes that are captured quantitatively through sensors (e.g. wearables), text (e.g. narrative), pictures, among any other data type. These raw data inputs are then pre-processed, analyzed through artificial intelligence/machine learning approaches to generate metrics. These metrics are stored in a database which is subsequently queried by the survey generation module at the time of survey.

TABLE 5
Example Applications to Various Clinical Indications

| | Example | | |
|---|---|---|---|
| Indication | Sensors | Algorithms | Metric |
| Knee Pain | wrist based | Computer vision/ | step count, knee |
| | accelerometers, | convolution | flexion angle, |
| | based capicitance | neural networks, | activity, distance |
| | sensors, computer | autoregression | walked. |
| | vision (e.g. cell | | |
| | phone camera, | | |
| | microsoft kinect | | |
| Scratch | wrist base | signal processing | frequency of |
| | accelerometer, | (fourier transform, | scratch, intensity |
| | adhesive skin | wavelets), recurrent | of scratch, skin |
| | thermometer, cell | neural networks | color, scratch area, |
| | phone picture | | skin temperature |
| Sleep | wrist base | signal processing | time to fall asleep, |
| | accelerometer, | (fourier transform, | total sleep time, |
| | leg capacitance, | wavelets), recurrent | cortical arousals, |
| | adhesive sternal | neural networks, | autonomic arousals |
| | multi-sensor, | computer vision, | |
| | off-body radio | convolutional | |
| | sensor, cell | neural nets | |
| | phone based | | |
| | accerometer | | |
| Quality | cell phone app | time series analysis, | cell phone use |
| of Life | tracking, wrist | graph theoretic | changes (app |
| | based sensors | network analyses | use changes), |
| | (accelerometry | | interactions |
| | PPG, gyroscopy), | | with friends, |
| | scales, GPS | | movement, weight |
| Nausea/ | sternal notch | time series analysis, | swallowing events, |
| Vomiting | adhesive sensor, | statistical modeling, | vomiting events, |
| | scale, wrist based | change detection | weight change, |
| | accelerometer | | eating frequency |

Table 5 provides a small example of the potential applications of this technology to 5 clinical indications. Notably a variety of sensors, algorithms, and metrics can be combined to generate the appropriate graphic for the improved survey. Critically, the ubiquity of wearable devices makes this method and system possible.

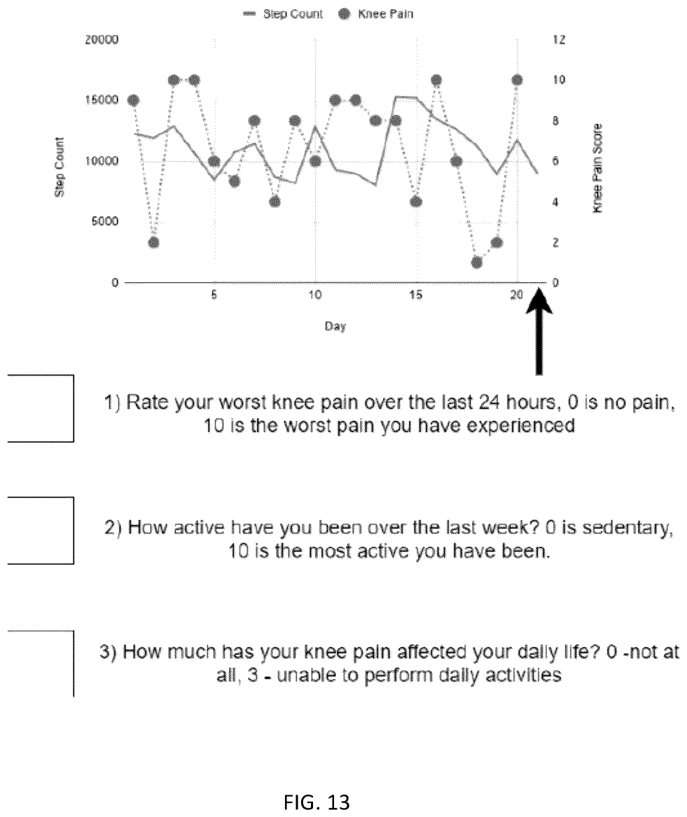

At the appropriate time (controlled by the protocol) a new survey instrument is produced that displays historical values of the survey instrument and customized data for the patient to reference. The questions that form the endpoints of interest for the study can also be displayed. Once the patient completes the survey the data are stored in the usual fashion. An example of the improved survey is provided in FIG. 13. The example survey of FIG. 13 considers a clinical trial that is evaluating a treatment effect on knee pain. Here we show the improved survey that displays the patient's previous evaluations of knee pain in addition to the number of steps taken over time. Providing these data to patients at the time of survey completion will enable more precise measurement of subjective treatment response. The bolded arrow denotes the time point at which the patient is to provide a rating of their knee pain. In practice the improved survey would show a different interface.

The result of such a system is the improvement and refinement of a patient's recall of their symptoms through the use of objective data that can help contextualize their response to a survey and ultimately the PRO. By providing objective data, we believe that interference from noisy memory processes can be improved and more clearly identify treatment effect.

In this example, a randomized clinical trial that is evaluating the effect of treatment on daily knee pain was considered. The proposed system can use an accelerometer (or other sensor) which can be processed to develop a metric of step counts. That information can be provided to the patient (along with their previous survey responses) graphically as part of the survey. This innovation provides a reference or anchor to help patients contextualize the responses being generated.

As disclosed herein, the improved survey has an overlay of previous PRO values (Knee pain ratings) with step counts. This allows the patient to recall and contextualize their previous ratings with an objective measure of step count. In the provided example, the patient would be queried to provide their rating of knee pain at the 3 week mark (denoted by the bold arrow).

Embodiments disclosed herein can improve the measurement of subjectively determined clinical trial measures specifically by more accurately measuring PROs. This is accomplished by providing patients historical and objective data with which they can anchor and/or contextualize their responses that are generated by noisy memory processes. By doing so, it is expected that subjective reports will be less biased and less variable which are well understood problems with human memory. It is anticipated that a principal use of this technology will be to improve the measurement accuracy of treatment effect (placebo or active).

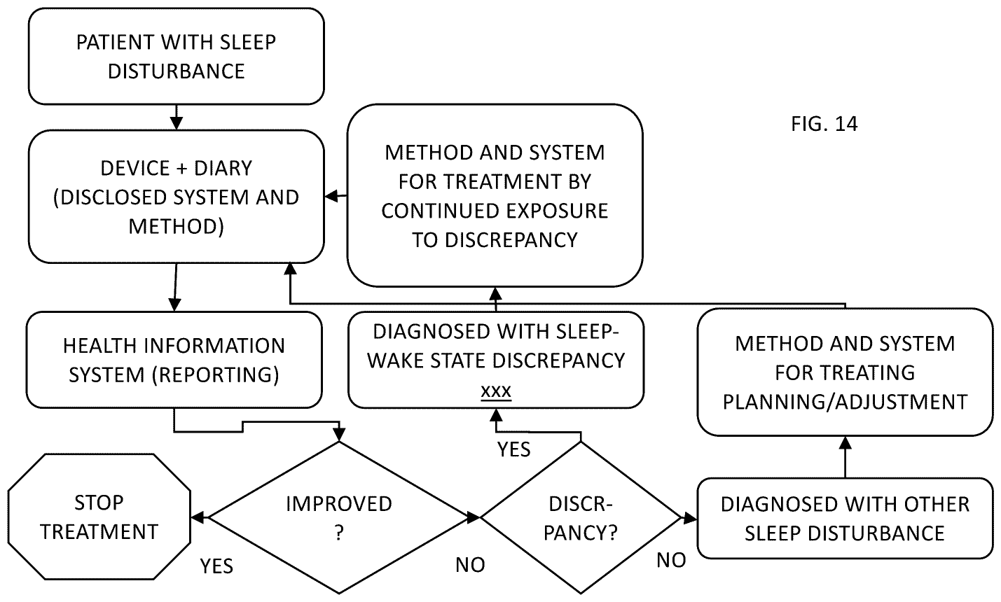

FIG. 14 illustrates a flow diagram of a method of use for health information reporting, treatment planning, diagnoses, and treatment in the context of sleep in accordance with embodiments of the present disclosure. Referring to FIG. 14, the method may be implemented by any example system shown herein such as the system 100 shown in FIG. 1. The method of FIG. 14 provides an in-situ example of the proposed system used for managing a patient with an unknown sleep disturbance.

With continuing reference to FIG. 14, utilizing the system, the patient collects data over a specified period through both subjective diary responses and objective measurements from the wearable device. Some example advantageous aspects of this system are the integration and presentation of data, alongside the patient's interaction with the diary, which are essential for effective data collection and treatment planning.

There are two principal outcomes highlighted by the system's use: The first outcome addresses sleep-wake state discrepancy, where there is a significant divergence between objective data from the wearable device and subjective diary entries. In such cases, the system's use is continued, leveraging the therapeutic potential of presenting objective data to correct misperceptions of sleep. The second outcome identifies another form of sleep disturbance, reinforcing the system's utility in diagnosing and managing a variety of sleep-related issues. Regardless of the specific diagnosis, the system enables patients to track their symptoms and progress effectively in a health information record.

With continuing reference to FIG. 14, the method and system presented, includes a wearable device, subjective surveys, and an integrated diary, is designed for the diagnosis, treatment planning, and treatment is applicable to a broader range of medical conditions. This underscores the system's comprehensive application in patient care, from initial assessment through ongoing management, highlighting its flexibility and adaptability.

The functional units described in this specification have been labeled as computing devices. A computing device may be implemented in programmable hardware devices such as processors, digital signal processors, central processing units, field programmable gate arrays, programmable array logic, programmable logic devices, cloud processing systems, or the like. The computing devices may also be implemented in software for execution by various types of processors. An identified device may include executable code and may, for instance, comprise one or more physical or logical blocks of computer instructions, which may, for instance, be organized as an object, procedure, function, or other construct. Nevertheless, the executable of an identified device need not be physically located together but may comprise disparate instructions stored in different locations which, when joined logically together, comprise the computing device and achieve the stated purpose of the computing device. In another example, a computing device may be a server or other computer located within a retail environment and communicatively connected to other computing devices (e.g., POS equipment or computers) for managing accounting, purchase transactions, and other processes within the retail environment. In another example, a computing device may be a mobile computing device such as, for example, but not limited to, a smart phone, a cell phone, a pager, a personal digital assistant (PDA), a mobile computer with a smart phone client, or the like. In another example, a computing device may be any type of wearable computer, such as a computer with a head-mounted display (HMD), or a smart watch or some other wearable smart device. Some of the computer sensing may be part of the fabric of the clothes the user is wearing. A computing device can also include any type of conventional computer, for example, a laptop computer or a tablet computer. A typical mobile computing device is a wireless data access-enabled device (e.g., an iPHONE® smart phone, an iPAD® device, smart watch, or the like) that is capable of sending and receiving data in a wireless manner using protocols like the Internet Protocol, or IP, and the wireless application protocol, or WAP. This allows users to access information via wireless devices, such as smart watches, smart phones, mobile phones, pagers, two-way radios, communicators, and the like. Wireless data access is supported by many wireless networks, including, but not limited to, Bluetooth, Near Field Communication, CDPD, CDMA, GSM, PDC, PHS, TDMA, FLEX, REFLEX, iDEN, TETRA, DECT, DataTAC, Mobitex, EDGE and other 2G, 3G, 4G, 5G, and LTE technologies, and it operates with many handheld device operating systems, such as EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone or smart watch that operates over GPRS (General Packet Radio Services), which is a data technology for GSM networks or operates over Near Field Communication e.g. Bluetooth. In addition to a conventional voice communication, a given mobile device can communicate with another such device via many different types of message transfer techniques, including Bluetooth, Near Field Communication, SMS (short message service), enhanced SMS (EMS), multi-media message (MMS), email WAP, paging, or other known or later-developed wireless data formats. Although many of the examples provided herein are implemented on smart phones, the examples may similarly be implemented on any suitable computing device, such as a computer.

An executable code of a computing device may be a single instruction, or many instructions, and may even be distributed over several different code segments, among different applications, and across several memory devices. Similarly, operational data may be identified and illustrated herein within the computing device, and may be embodied in any suitable form and organized within any suitable type of data structure. The operational data may be collected as a single data set, or may be distributed over different locations including over different storage devices, and may exist, at least partially, as electronic signals on a system or network.

The described features, structures, or characteristics may be combined in any suitable manner in one or more embodiments. In the following description, numerous specific details are provided, to provide a thorough understanding of embodiments of the disclosed subject matter. One skilled in the relevant art will recognize, however, that the disclosed subject matter can be practiced without one or more of the specific details, or with other methods, components, materials, etc. In other instances, well-known structures, materials, or operations are not shown or described in detail to avoid obscuring aspects of the disclosed subject matter.

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. For example, exemplary embodiments are intended to cover all software or computer programs capable of enabling processors to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed below.

In accordance with the exemplary embodiments, the disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl, or other suitable programming languages.

As referred to herein, a computer network may be any group of computing systems, devices, or equipment that are linked together. Examples include, but are not limited to, local area networks (LANs) and wide area networks (WANs). A network may be categorized based on its design model, topology, or architecture. In an example, a network may be characterized as having a hierarchical internetworking model, which divides the network into three layers: access layer, distribution layer, and core layer. The access layer focuses on connecting client nodes, such as workstations to the network. The distribution layer manages routing, filtering, and quality-of-server (QoS) policies. The core layer can provide high-speed, highly-redundant forwarding services to move packets between distribution layer devices in different regions of the network. The core layer typically includes multiple routers and switches.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed is:

1. A method comprising:
at a computing device comprising a health data manager:
receiving objective health data of a subject;
receiving a first set of information about the subject's health, wherein first set of information was conveyed by the subject;
presenting, via a user interface, the objective health data to the subject;
receiving, subsequent to presentation of the objective health data to the subject, a second set of information about the subject's health, wherein the second set of information was conveyed by the subject;
analyzing the objective health data, the first set of information, and the second set of information;
determining a treatment plan, diagnosis, and/or health information based on the analysis; and
presenting, via the user interface, the treatment plan, treatment, diagnosis, and/or health information.

2. The method of claim 1, wherein receiving the objective health data comprises using one or more physical sensors to acquire the objective health data from the subject.

3. The method of claim 2, wherein the one or more physical sensors comprise polysomnography equipment, a wearable device, an accelerometer, and/or a motion sensor.

4. The method of claim 1, wherein the first set of information and the second set of information comprise total sleep time and/or confidence in a physical parameter.

5. The method of claim 1, wherein the objective health data comprises sleep onset latency, total sleep time, wake after sleep onset, and/or sleep efficiency.

6. The method of claim 1, wherein analyzing the objective health data comprises determining a variance and/or mean of the objective health data, the first set of information, and the second set of information.

7. The method of claim 6, further comprising presenting, via the user interface, the variance and/or mean.

8. The method of claim 1, wherein the received, objective health data of the subject includes data acquired about a predetermined health characteristic of the subject within a predetermined period of time, and
wherein the first set of information about the subject's health is associated with the subject's conveyance of opinion about the predetermined health characteristic of the subject within the predetermined period of time.

9. The method of claim 8, further comprising:
comparing the data about the predetermined health characteristic with the subject's conveyed opinion about the predetermined health characteristic; and
presenting, via the user interface, the comparison of the data about the predetermined health characteristic with the subject's conveyed opinion about the predetermined health characteristic.

10. The method of claim 1, wherein the received, objective health data of the subject includes data acquired about a predetermined health characteristic of the subject within a plurality of different periods of time,
wherein the first set of information about the subject's health is associated with the subject's conveyance of opinion about the predetermined health characteristic of the subject within the plurality of different periods of time, and
wherein the method further comprises presenting, via the user interface, the data about the predetermined health characteristic and the subject's conveyed opinion about the predetermined health characteristic.

11. A computing device comprising:
a user interface; and
a health data manager configured to:
receive objective health data of a subject;
receive a first set of information about the subject's health, wherein first set of information was conveyed by the subject;
present, via the user interface, the objective health data to the subject;
receive, subsequent to presentation of the objective health data to the subject, a second set of information about the subject's health, wherein the second set of information was conveyed by the subject;
analyze the objective health data, the first set of information, and the second set of information;
determine a treatment plan, diagnosis, and/or health information based on the analysis; and
present, via the user interface, the treatment plan, diagnosis, treatment, and/or health information.

12. The computing device of claim 11, wherein the health data manager is configured to use one or more physical sensors to acquire the health data from the subject.

13. The computing device of claim 12, wherein the one or more physical sensors comprise polysomnography equipment, a wearable device, an accelerometer, and/or a motion sensor.

14. The computing device of claim 11, wherein the first set of information and the second set of information comprise total sleep time and/or confidence in a physical parameter.

15. The computing device of claim 11, wherein the objective health data comprises sleep onset latency, total sleep time, wake after sleep onset, and/or sleep efficiency.

16. The computing device of claim 11, wherein the health data manager is configured to determine a variance and/or mean of the objective health data, the first set of information, and the second set of information.

17. The computing device of claim 16, wherein the health data manager is configured to utilize the user interface to present the variance and/or mean.

18. The computing device of claim 11, wherein the received, objective health data of the subject includes data acquired about a predetermined health characteristic of the subject within a predetermined period of time, and
wherein the first set of information about the subject's health is associated with the subject's conveyance of opinion about the predetermined health characteristic of the subject within the predetermined period of time.

19. The computing device of claim 18, wherein the health data manager is configured to:
compare the data about the predetermined health characteristic with the subject's conveyed opinion about the predetermined health characteristic; and
utilize the user interface to present the comparison of the data about the predetermined health characteristic with the subject's conveyed opinion about the predetermined health characteristic.

20. The computing device of claim 11, wherein the received, objective health data of the subject includes data acquired about a predetermined health characteristic of the subject within a plurality of different periods of time,
wherein the first set of information about the subject's health is associated with the subject's conveyance of opinion about the predetermined health characteristic of the subject within the plurality of different periods of time, and
wherein the health data manager is configured to present, via the user interface, the data about the predetermined health characteristic and the subject's conveyed opinion about the predetermined health characteristic.

21. A system comprising:
one or more physical sensors configured to acquire objective health data from a subject; and
a computing device comprising:
a user interface; and
a health data manager configured to:
receive the objective health data of the subject from the one or more physical sensors;
receive a first set of information about the subject's health, wherein first set of information was conveyed by the subject;
present, via the user interface, the objective health data to the subject;
receive, subsequent to presentation of the objective health data to the subject, a second set of information about the subject's health, wherein the second set of information was conveyed by the subject;
analyze the objective health data, the first set of information, and the second set of information;
determine a treatment plan, diagnosis, and/or health information based on the analysis; and
present, via the user interface, the treatment plan, diagnosis, treatment, and/or health information.
